(19) United States
(12) Patent Application Publication
(10) Pub. No.: US 2014/0275518 A1
(43) Pub. Date: Sep. 18, 2014

(54) L-GLUCOSE PRODUCTION FROM L-GLUSOSE/L-MANNOSE MIXTURES USING SIMULATED MOVING BED SEPARATION

(71) Applicant: OROCHEM TECHNOLOGIES, INC., Lombard, IL (US)

(72) Inventors: Anil R. Oroskar, Oak Brook, IL (US); Rakesh Vilraman Nair Rema, Downers Grove, IL (US)

(73) Assignee: OROCHEM TECHNOLOGIES, INC., Lombard, IL (US)

(21) Appl. No.: 13/831,089

(22) Filed: Mar. 14, 2013

(51) Int. Cl. C13K 1/00

(52) U.S. Cl. CPC C13K 1/00
USPC 536/125

(57) ABSTRACT

Disclosed is a process for the production of L-glucose from a mixture of L-mannose and L-glucose to provide a high purity L-glucose product. More particularly, the invention relates to a process for the isomerization of mixtures of L-mannose and L-glucose to favor the epimerization or transformation of the L-mannose into L-glucose combined with the selective removal of impurities and the selective separation of L-glucose by a multi-stage simulated moving bed SMB separation process integrating ion exclusion and isomer separation. The process is useful for providing a simplified and economic continuous processing route to providing pure L-glucose from mixtures of L-glucose and L-mannose in the presence of inorganic and organic salts and other sugars such as L-arabinose. L-glucose is useful as a sweetener, a laxative and as a therapeutic agent.

### FIELD OF THE INVENTION

This invention concerns generally with a process for the production of L-glucose from saccharide mixtures thereof. More specifically, the invention is a process for the recovery of L-glucose from mixtures comprising L-glucose and L-mannose. More particularly, it relates to a process for the recovery of high purity L-glucose from comprising L-glucose and L-mannose and the use of a multi-stage separation scheme based on simulated moving bed (SMB) separation. L-glucose is effective for medical applications.

### BACKGROUND

L-Glucose is an organic compound with formula C₆H₁₂O₆ or H—(C═O)—(CHOH)₅—H, which is one of the aldohexose monosaccharides. L-glucose has a structure which is generally represented as follows:

[chemical structure]

L-glucose is the L-isomer of glucose; that is, it is the enantiomer, or optical isomer, of D-glucose. Prefixes D- and L- in a monosaccharide name identify one of two isomeric forms. L-glucose does not occur naturally in higher living organisms, cannot be used by living organisms as source of energy. L-glucose has been shown to be effective as a colon cleanser for patients preparing to have a colonoscopy.

Mannose is a sugar monomer of the aldohexose series of carbohydrates. Mannose is a C-2 epimer of glucose and has an L-isomer and a D-isomer. The structure of L-mannose is shown below:

[chemical structure]

L-Mannose is important in human metabolism, especially in the glycosylation of certain proteins (i.e., the enzymatic process that attaches glycans to proteins).

A variety of methods exist for separating polar organic substances from ionic substances. Many of these methods require multiple purification steps and do not achieve complete separation. For example, U.S. Pat. Nos. 5,968,362 and 6,391,204 describe methods involving the use of an anionic exchange resin to remove heavy metals and acid from organic substances. However, these methods are not amenable to complete acid removal, nor do they allow for removal of inorganic and organic cations and anions simultaneously. Similarly, U.S. Pat. Nos. 5,538,637 and 5,547,817 describe methods for separating acids from sugar molecules. However, these methods are limited to separating acids and are not applied to the simultaneous removal of all forms of inorganic and organic cations and anions. Additionally, U.S. Patent Publication Nos. 2009100556707 and 200810041366 disclose using an ion exchange resin for separating first calcium sulfate then acids from sugar mixtures.

Improved methods are sought for the separation and production of high purity L-glucose from mixtures of inorganic and organic cations such as salts and other sugar molecules.

### SUMMARY

The present invention is based on the integration of multiple simulated moving bed separation zones having multiple extract and raffinate stream with an isomerization zone to provide a process which can convert L-mannose to L-glucose to enhance the concentration of L-glucose and provide for the essentially complete conversion of L-mannose to L-glucose within the overall process, while recovering at least a portion of the mobile phase desorbent streams to minimize operating and raw material costs.

In one embodiment, the invention is a process for the production of high purity L-glucose product from a mixed feed stream comprising L-glucose, L-mannose, salts and other sugars. The process comprises passing the mixed feed stream at a mixed feed temperature and a first mobile phase stream comprising water to an ion exclusion SMB zone comprising a plurality of ion exclusion beds. Each of the ion exclusion beds contains an ion exclusion stationary phase agent comprising a strong acid sodium exchange resin. The ion exchange stationary phase agent is selective for the adsorption of L-mannose and L-glucose and other sugars. The ion exclusion SMB zone is operated in an ion exclusion cycle to provide a first extract stream having a reduced concentration of salts and an initial concentration of L-glucose on a total sugar basis and comprising L-glucose, L-mannose, other sugars and water, a first primary raffinate stream comprising water and salts, and a first secondary raffinate stream comprising water. The first extract stream is admixed with a second secondary extract stream comprising L-mannose and water to provide an evaporization zone feed stream, and the evaporization zone feed stream is passed to an evaporization zone to provide an evaporization zone effluent stream comprising water, L-glucose and L-mannose. The evaporization zone effluent has a reduced concentration of water relative to the evaporization zone feed stream. The evaporization zone effluent stream is passed to an isomerization zone to at least partially transform a portion of the L-mannose into L-glucose to provide an isomerization zone effluent stream comprising L-glucose, L-mannose, and water. The isomerization zone effluent stream has a concentration of L-glucose on a total sugar basis which is enhanced relative to said initial concentration of L-glucose in the first extract stream. The isomerization zone effluent stream and a second mobile phase stream comprising water are passed to a second SMB zone comprising a plurality of glucose separation beds. Each of the glucose separation beds contains a glucose stationary phase agent comprising a strong acid calcium exchange resin which is selective for the adsorption of L-glucose in a glucose adsorption cycle at effective glucose/mannose separation conditions to provide a second primary extract stream comprising L-mannose, a second secondary extract stream comprising L-arabinose, a second primary raffinate stream comprising high purity L-glucose, and a second secondary raffinate stream comprising water. The second primary extract stream is admixed with the first extract stream in step (b). At least a portion of the first secondary raffinate stream comprising water is returned to the ion exclusion SMB zone to provide at least a portion of the first mobile phase stream. At least a portion of the second secondary raffinate stream comprising water is returned to the second SMB zone. to provide at least a portion of the second mobile phase stream. The second primary raffinate stream is passed to an L-glucose recovery zone comprising distillation or evaporization to provide the high purity L-glucose product.

### BRIEF DESCRIPTION OF THE DRAWINGS

FIG. 1 is a schematic process flow diagram illustrating one embodiment of the invention employing a single isomerization zone and 2 SMB separation zones.

FIG. 2 is a schematic process flow diagram illustrating one embodiment of the invention illustrating the operation of two eight-adsorbent bed SMB separation zones.

FIG. 3 is a schematic process flow diagram illustrating one embodiment of the invention illustrating the operation of two fifteen-adsorbent bed SMB separation zones.

### DETAILED DESCRIPTION OF THE INVENTION

In the separation processes of the instant invention, chromatographic separation systems are used to separate a mixture of L-mannose and L-glucose from salts, and to separate a high purity L-glucose product from mixtures comprising L-mannose, L-glucose, and impurities such as L-arabinose and salts. The chromatographic separator may include a batch type operation or the generally more efficient simulated moving bed operation, and operated using continuous internal recirculation. Examples of simulated moving bed processes are disclosed, for instance, in U.S. Pat. No. 6,379,554 (method of displacement chromatography); U.S. Pat. No. 5,102,553 (time variable simulated moving bed process), U.S. Pat. No. 6,093,326 (single train, sequential simulated moving bed process); and U.S. Pat. No. 6,187,204 (same), each of the contents of the entirety of which is incorporated herein by this reference.

The SMB system of the current invention was arranged for maximum selectivity. The simulated moving bed operation is achieved by use of a plurality of adsorbent beds connected in series and a complex valve system, whereby the complex valve system facilitates switching at regular intervals the feed entry in one direction, the mobile phase desorbent entry in the opposite direction, while changing the extract and raffinate takeoff positions as well. The SMB system is a continuous process. Feed enters and extract and raffinate streams are withdrawn continuously at substantially constant compositions. The overall operation is equivalent in performance to an operation wherein the fluid and solid are contacted in a continuous countercurrent manner, without the actual movement of the solid, or stationary phase adsorbent.

The operation of the SMB system is carried out at a constant temperature within the adsorbent bed. Preferably, the SMB zones of the present invention operate at an SMB temperature of about 40° C. to about 75° C. More preferably, the SMB zones of the present invention operate at an SMB temperature of between about 65° C. to about 70° C. The feed stream is introduced and components are adsorbed and separated from each other within the adsorbent bed. The feed to the SMB zone can be introduced to the SMB zone at a feed temperature of from room temperature (25° C.) to about 70° C. In order to avoid possible caramelization of the feed stream in commercial size plants, the feed may be stored at any feed storage temperature and then passed through a heat exchange zone to provide the feed stream at the appropriate SMB temperature, rather than holding a feed storage tank at the required feed temperature. Caramelization is a culinary phenomenon that occurs when carbohydrates like glucose are heated to temperatures of 160° C. or higher, causing them to turn brown. A separate liquid, the mobile phase desorbent, is used to counter currently displace the feed components from the pores of the stationary phase adsorbent. The mobile phase desorbent may be introduced to the SMB zone at a mobile phase temperature of 40-75° C. More preferably, the mobile phase desorbent may be introduced to the SMB zone at a mobile phase temperature of 60-75° C. During the SMB cycle of the present invention, adsorbent beds are advanced through a desorption zone, a rectification zone, an adsorption zone, and at least one regeneration zone. The description of the SMB cycle as a 2-3-3 cycle means that in the cycle, 2 adsorbent beds are in the desorption zone, 3 adsorbent beds are in the rectification zone, and 3 adsorbent beds are in the adsorption zone. A novel aspect of the present invention in the first SMB zone, or ion exclusion zone, is the use of two regeneration zones to provide a first primary raffinate and a first secondary raffinate, whereby the first secondary raffinate can be returned to the first SMB zone to provide at least a portion of the first mobile phase desorbent. In the first SMB zone, the primary raffinate is passed to waste water recovery and the secondary raffinate is sufficiently pure to be returned or recycled to the first SMB zone as the mobile phase stream, this reducing the overall requirement for mobile phase and eliminating a separate mobile phase recovery step in the overall process. In the second SMB zone, or glucose separation SMB zone, there is a primary and secondary extract stream, and a primary and a secondary raffinate stream. The second secondary extract stream can provide an L-arabinose byproduct stream. The second primary extract stream comprises mostly L-mannose which can be combined with the first primary extract stream and the combined stream can be isomerized after evaporation to improve the overall recovery and purity and yield of high purity L-glucose.

### Stationary Phase

In one embodiment, the present invention comprises two SMB zones. A first SMB zone, or ion exclusion SMB zone, comprises a first stage adsorbent or ion exclusion stationary phase agent which is effective for removing salts in an ion exclusion step. The second SMB zone is effective for the separation of L-glucose from L-mannose and other sugars such as L-arabinose. In the first SMB zone, a first SMB stationary phase agent, or ion exclusion stationary phase agent comprising a strong acid sodium exchange resin has been found to be effective. The ion exclusion cycle for an 8 ion exclusion bed SMB of the first SMB zone comprises a 2-3-2-1 cycle having 2 ion exclusion beds in a desorption zone, 3 ion exclusion beds in a rectification zone, 2 ion exclusion beds in an adsorption zone, and 1 ion exclusion bed in a first regeneration zone. In a 15 bed ion exclusion SMB zone, the SMB cycle comprises a 4-5-5-1 cycle. In the second SMB zone it is preferred that a second SMB zone stationary phase agent be a strong acid calcium exchange resin for the separation of L-glucose from L-mannose. The second SMB cycle for an 8 adsorbent bed SMB of the second SMB zone comprises a 1-1-3-2-1 cycle having 1 adsorbent bed in a desorption zone, 1 adsorbent bed in a second desorption zone, 3 adsorbent beds in a rectification zone, and 2 adsorbent bed in an adsorption zone and 1 adsorbent bed in a first regeneration zone and 1 adsorbent bed in a second regeneration zone. In a 15 bed adsorbent SMB zone, the SMB cycle comprises a 2-2-5-5-1 cycle.

The calcium exchanged resins used in the glucose separation SMB zone may be made by the process described in U.S. Pat. No. 4,444,961, which provides very uniform spherical size polymeric beads. Preferably, the stationary phase adsorbent will have an average particle size of from 220 microns to about 350 microns and the resin will have a cross link percentage of from about 4 to about 8 percent. More preferably, the glucose separation stationary phase agent will have an average particle size of from 220 microns to about 350 microns and the resin will have a cross link percentage of from about 6 to about 8 percent. U.S. Pat. No. 4,444,961 is hereby incorporated in its entirety by reference. In some cases, the resin may be available in the hydrogen form, and the resin may be exchanged with Ca²⁺ or Na⁺ or K⁺ ions. Alternatively, the resin may be exchanged with multiple ions in a single solution in a ratio calculated or experimentally determined to exchange the respective ions in the desired ratio. Exchange methods are well known to those of ordinary skill in the art and are suitable for the resins of this invention. The preferred SMB stationary phase agent for glucose separation in the second SMB zone is a strong acid cation calcium exchange resin such as DOWEX 99CA/320 (Available from The Dow Chemical Company, Midland, Mich.), or other such resins as Rohm and Haas 1310 and 1320 resins, PUROLITE PCR resins (Available from Purolite, Bala Cynwyd, Pa.), and other DOWEX monosphere chromatographic resins. Other such resins include UBK555 (Mitsubishi Chemical Co., Carmel Ind.).

### Mobile Phase Desorbent

Water or deionized water is used as the mobile phase eluent for the SMB zones. Other eluents that perform functions the same as or similar to water known to those of ordinary skill in the art are also contemplated herein.

### L-Glucose Isomerization

The isomerization of the L-mannose can be carried out by any conventional means such as described in U.S. Pat. No. 4,581,447, wherein the conversion of L-arabinose to a mixture of L-glucocyanohydrin and L-mannocyanohydrin by the reaction of a cyanide source with L-arabinose. Suitable cyanide sources include cyanide salts, such as those of alkali metals, with sodium and potassium cyanide being favored, as well as other water soluble salts furnishing cyanide ion, and hydrocyanic acid or hydrogen cyanide. An essential feature of the '447 patent is that during the course of the reaction the pH is maintained between about 7.0 and about 9.0, most preferably between about 7.8 and 8.2. The next step is the selective hydrogenation of the cyanohydrins to their corresponding imines with subsequent hydrolysis of the imines to their corresponding aldehydes under conditions where the resulting aldehydes are not hydrogenated. As disclosed in the U.S. Pat. No. 4,581,447 the composition of the resulting hydrogenation mixture is about a 60:40 mixture of L-mannose:L-glucose in a total yield up to about 85% based on L-arabinose.

### DETAILED DESCRIPTION OF THE DRAWINGS

According to one embodiment of the invention and with reference to FIG. 1, a mixed feed stream comprising L-mannose and L-glucose, and other sugars (such as L-arabinose), and salts in line 12 and a first mobile phase desorbent stream comprising water in line 10 are passed to an ion exclusion SMB zone, or first SMB zone 101 for salt removal to provide a first primary extract stream in line 18 comprising L-mannose, L-glucose, a reduced amount of other sugars and salts in water, a first secondary raffinate stream in line 14 comprising water, and a first primary raffinate stream in line 16 comprising water, and salts such as sodium sulfate and ammonium sulfate. The first primary raffinate stream in line 16 is passed to waste water recovery (not shown). The first secondary raffinate stream in line 14 consisting essentially of water (99.99 to 100 wt-%) is recycled and at least a portion of the first secondary raffinate stream in line 14 is combined with the first mobile phase desorbent stream in line 10 (not shown) to conserve mobile phase desorbent. The first primary extract stream in line 18 is combined with a second primary extract stream in line 30 comprising L-mannose and water to provide a combined extract stream in line 20 and the combined extract stream is passed to a first evaporization zone 102. The first evaporization zone 102 can employ any conventional evaporization or vacuum distillation technique to remove at least a portion of the water from the combined extract stream in line 20 to provide a first water stream in line 22, and an isomerization zone feed stream in line 24. The first water stream is passed to waste water recovery (not shown). The evaporization zone effluent stream will comprise a reduced concentration of water relative to the evaporization zone feed stream in line 24. Preferably, the isomerization zone feed stream in line 24 has a Brix value of from about 15 to about 20. In aqueous sugar solutions, the density of the aqueous sugar solution is typically expressed in Brix; for example, a 20 Brix solution is a measurement of the dissolved sugar-to-water mass ratio of a liquid, where 20 Brix is equivalent to 20 grams of sugar in 80 grams of water), The isomerization zone feed stream in line 24 is passed to an isomerization zone 103 for the isomerization of L-mannose to L-glucose according to any method such as the method disclosed hereinabove to provide an isomerization effluent stream in line 26, comprising L-mannose, L-glucose, and water. The isomerization effluent stream in line 26 and a second mobile phase desorbent stream in line 28 are passed to a second SMB zone 104. The second SMB zone comprises a plurality of glucose separation beds, each glucose separation bed contains a second stationary phase agent selective for the separation of L-glucose, comprising a calcium exchanged strong acid resin particle having a particle size of from about 300 to about 320 microns. The second SMB zone 104 operates according to a second SMB cycle, referred to herein as a 1-1-3-2-1 SMB cycle, to provide the second primary extract stream in line 30 comprising water and L-mannose which is recycled to the isomerization zone 103 as described above, a second secondary extract stream in line 30, a second secondary raffinate stream in line 34 comprising water, and a second primary raffinate stream in line 32 comprising L-glucose and water. The second secondary extract stream in line 36 is essentially salt free and comprises L-mannose, L-glucose, and L-arabinose. The second secondary extract stream comprises a major portion of L-arabinose and less than 10 wt-% of L-glucose on a total sugar basis. More preferably, the second secondary extract stream in line 36 comprises less than about 5 wt-% L-glucose on a total sugar basis. The second secondary extract stream may be passed to L-arabinose recovery (not shown). The second primary raffinate stream in line 32 is passed to a second evaporization zone 106 to provide a second evaporization water stream in line 38 and an evaporated second raffinate stream in line 40. The evaporated second raffinate stream in line 40 comprises substantially pure L-glucose (i.e., 90, 93, 95, 96, 97, 98, 99, 99.5 wt % of L-glucose, based on total sugar), and the remainder being a minor portion of L-mannose). The evaporated second raffinate stream in line 40 is passed to a crystallization and drying zone 105 to provide a high purity L-glucose product stream in line 42. The high purity L-glucose stream may be in the form of a syrup, a granular or a crystalline product as processed by any conventional manner (not shown).

Depending on the original quality of the high purity L-glucose material, the second primary raffinate stream from the second SMB zone may require further purification, clean-up or polishing, usually to remove residual color. Addition of final polishing represents separate embodiments of our invention. If desired, it is recommended that the optional polishing step include one or more of the following known color removal methods: ion exchange, absorption, chemical treatment, carbon treatment or membrane treatment. Chemical treatment can include the addition of oxidizing agents, such as hydrogen peroxide wherein 0.1% to 0.15% on weight or equivalent conventionally recommended dosage. An example of membrane treatment is the employment of nano-filtration membranes which can remove small remaining colored compounds.

Evaporation of, or water removal from the L-glucose product stream or the second primary raffinate stream removed from the second SMB zone, will be unnecessary when low amounts of dissolved solids are present and it is desired to, e.g., send to water treatment or water disposal facilities. Optionally, one of ordinary skill in the art may desire, e.g., to evaporate such streams for commercial reasons to concentrate remaining solids.

Further purification methods may include filtration, evaporation, distillation, drying, gas absorption, solvent extraction, press extraction, adsorption, crystallization, and centrifugation. Other purification methods may include further chromatography according to this invention utilizing batch, simulated moving bed (including continuous, semi-continuous, or sequential), such simulated moving bed utilizing more than one loop, utilizing more than one profile, less than one profile, or combinations of any of the forgoing as will be appreciated for application with this invention by those of ordinary skill in the art after reading this disclosure. In addition, further purification can include combinations of any of the forgoing, such as for example, combinations of different methods of chromatography, combinations of chromatography with filtration, or combinations of membrane treatment with drying.

In one other embodiment of the present invention, the first and second SMB zones each contain 8 adsorption beds. In the first SMB zone, SMB-1, as shown in FIG. 2, the adsorption beds of the first SMB zone are serially connected and numbered from left to right from 181 to 188. Each bed has a top and a bottom, and each bed in the first SMB zone contains a first stationary phase agent. The adsorption beds 181-188 are arranged such that in accordance with the first SMB cycle, the first mobile phase desorbent stream in line 201 comprising deionized water at a desorbent temperature of 40-70° C. is introduced to the top of adsorbent bed 181 and continues to cascade in a serial manner through all of the adsorption beds 181-188, flowing to the top of a first adsorption bed, such as adsorption bed 181, through adsorption bed 181, and the first mobile phase desorbent is withdrawn from the bottom of the first adsorption bed, adsorption bed 181, and then passed to the top of the next adsorption bed, adsorption bed 182. This serial cascade of the first mobile phase desorbent is continued through a first desorption zone (181 to 182), a first rectification zone (183 to 185), and a first adsorption zone (186 to 187) until the first secondary raffinate stream, or recovered first mobile phase desorbent stream is recovered and is withdrawn in line 201′ from the last adsorption bed, adsorption bed 188 being in isolation. All or a portion of the first secondary raffinate stream, or recovered first mobile phase desorbent may be returned to provide at least a portion of the first mobile phase desorbent in line 201 (not shown). According to the 2-3-2-1 SMB cycle of the first SMB zone, the first mixed feed stream comprising L-sugars and salts at a feed temperature of about 70° C. is passed to the top of adsorption bed 186 in line 202, the first raffinate stream comprising salt, water, and a small amount of L-sugars in line 203 is withdrawn from the bottom of adsorption bed 187, and a first extract stream comprising the L-sugars and a small amount of salt is withdrawn from the bottom of adsorption bed 182 in line 204. Similarly, with reference to the second SMB zone, SMB-2, shown in FIG. 2, the second SMB zone contains 8 serially connected adsorbent beds (numbered from left to right 281-288). Each of the adsorbent beds in the second SMB zone contains a second stationary phase agent and has a top and a bottom. The adsorption beds 281-288 are arranged such that in accordance with the second SMB cycle, the second mobile phase desorbent stream in line 208 comprising deionized water at a desorbent temperature of 40-70° C. is introduced to the top of 281 and continues to cascade in a serial manner through all of the adsorption beds 281-288, flowing from the top of a first adsorption bed, such as adsorption bed 281, through adsorption bed 288, and the first mobile phase desorbent is withdrawn from the bottom of the first adsorption bed, adsorption bed 281, and then passed to the top of the next adsorption bed, adsorption bed 282. This serial cascade of the second mobile phase desorbent is continued until the recovered second mobile phase desorbent stream is recovered and is withdrawn from the last adsorption bed in the second SMB serial arrangement, adsorption bed 288, in line 208′. All or a portion thereof of the recovered second mobile phase desorbent may be returned to provide at least a portion of the first mobile phase desorbent in line 208 (not shown). According to the 1-1-3-2-1 SMB cycle of the second SMB zone which differs slightly from the SMB cycle of the first SMB zone, there are two extract streams: a second primary extract stream, in line 211, comprising mainly L-mannose; and, a second secondary extract stream, in line 210, comprising mainly L-arabinose. The second secondary extract stream in line 210 can be recovered as a byproduct L-arabinose stream following water removal, and the second primary extract stream in line 211 is combined with the first extract stream in line 204 and passed to an evaporization or distillation zone to remove at least a portion of the mobile phase desorbent prior to passing an evaporated combined extract stream to an isomerization or epimerization zone (see FIG. 1, not shown in FIG. 2) to provide an isomerate stream having an enhanced L-glucose concentration by conversion of at least a portion of the L-mannose. The isomerization zone effluent stream is passed to the second SMB zone at a second SMB feed temperature of about 65-70° C. is passed to the top of adsorption bed 286 via line 207, the second primary raffinate stream comprising L-glucose in line 209 is withdrawn from the bottom of adsorption bed 287, and a first primary extract stream in line 211 is withdrawn from the bottom of adsorption bed 282, and the second secondary extract stream is withdrawn from adsorption bed 281 in line 210.

With reference to FIG. 3, a further embodiment of the present invention based on a 15 adsorbent bed arrangement for the first SMB zone and the second SMB zone. The first and second SMB zones each contain 15 adsorption beds. In the first SMB zone, as shown in FIG. 3, the adsorption beds of the first SMB zone are serially connected and numbered from left to right from 1-151 to 1-1515. Each bed has a top and a bottom, and each bed contains a first stationary phase agent, or ion exclusion stationary phase agent. The adsorption beds of the second SMB zone are serially connected and numbered from left to right from 2-151 to 2-1515. Each bed has a top and a bottom, and each bed contains a second stationary phase agent. With reference to the first SMB zone, the first mobile phase desorbent stream is introduced to the top of adsorbent bed 1-151 in line 401 and a recovered first mobile phase adsorbent stream is collected from the bottom of adsorbent bed 1-1515 in line 401′. The mixed feed stream is introduced to the top of adsorbent bed 1-1510, a first primary raffinate stream is withdrawn from the bottom of adsorbent bed 1-1514 via line 403, and a first extract stream is withdrawn from adsorbent bed 1-154 in line 404. Referring to the second SMB zone, the second mobile phase desorbent stream is introduced to the top of adsorbent bed 2-151 in line 408 and a recovered second mobile phase adsorbent stream, or second secondary raffinate stream, is collected from the bottom of adsorbent bed 2-1515 in line 408′. The isomerization zone effluent stream is passed to the second SMB zone at a second SMB feed temperature of about 65-70° C. to the top of adsorption bed 22-1510 via line 407, the second raffinate stream comprising L-glucose in line 409 is withdrawn from the bottom of adsorption bed 2-1513, and a first primary extract stream in line 411 is withdrawn from the bottom of adsorption bed 2-154, and the second secondary extract stream is withdrawn from adsorption bed 2-152 in line 410.

The following examples are provided to illustrate the present invention. These examples are shown for illustrative purposes, and any invention embodied therein should not be limited thereto.

### EXAMPLES

All purities or recovery values are generally expressed in terms of the total sugar content of the product or stream. In general, a high purity stream will comprise from 90 to 99 wt-% of the key component based on the total sugar in the product or stream. Similarly, recoveries are expressed in terms of recovery based on the total sugar content.

### Example 1

### Material Balance

A high purity L-glucose product was recovered from a mixture of L-sugars using the process of the present invention. Results are shown herein for a 50 MTA production of L-glucose. The mixture of L-sugars had the composition shown in Table 1.

TABLE 1
Composition of Feed Stream to First SMB Zone

| Component | Wt-% |
|---|---|
| L-Mannose | 7.34 |
| L-Glucose | 3.95 |
| L-Arabinose | 0.26 |
| Na₂SO₄ | 15.78 |
| (NH₄)₂SO₄ | 4.14 |
| Pri Amines | 0.72 |
| Related Monosaccharides | 0.02 |
| Aldonic Acid | 0.24 |
| Water | 67.54 |
| Total | 100.00 |

According to the process as described hereinabove in FIG. 1, the mixture of L-sugars or mixed feed stream was passed to a first SMB zone, or ion exclusion zone containing 8 ion exclusion beds in a 2-3-3 configuration, each adsorbent bed containing sodium exchange resin in the form of a spherical particle having a particle size of from 300 to 320 microns. The feed stream was passed to the first SMB zone at a feed rate of 2414 kg/day and a first mobile phase desorbent stream comprising water at a first mobile phase temperature of 70-75° C. at a first desorbent rate of 25694 kg/day was passed to the first SMB zone. The SMB zone consisted of 8-203 mm by 1219 mm cylindrical adsorbent beds filled with the sodium exchange resin adsorbent and operated in a 2-3-3 SMB cycle at a cycle time of less than about 15 minutes to provide a first raffinate stream, a first primary extract, a first secondary extract stream, and a first secondary raffinate stream or first desorbent effluent stream. The mobile phase consisted of water, and at least a portion of the first desorbent effluent stream was recycled and combined with the first mobile phase desorbent stream to minimize mobile phase desorbent use. Table 2 shows the composition of the first feed, first extract stream and first raffinate stream.

TABLE 2
Composition of First SMB Streams

| | Feed | Extract | Raffinate |
|---|---|---|---|
| Component | Wt-% | Wt-% | Wt-% |
| L-Mannose | 7.34 | 4.12 | 0.04 |
| L-Glucose | 3.95 | 2.21 | 0.02 |
| L-Arabinose | 0.26 | 0.15 | 0 |
| Na₂SO₄ | 15.78 | 0 | 5.60 |
| (NH₄)₂SO₄ | 4.14 | 0 | 1.47 |
| Pri Amines | 0.72 | 0 | 0.26 |
| Related | 0.02 | 0.5 | 0 |
| Monosaccharides | | | |
| Aldonic Acid | 0.24 | 0 | 0 |
| Water | 67.54 | 93.50 | 92.61 |
| Total | 100.00 | 100.00 | 100.00 |

The first raffinate stream was passed to a waste water recovery zone at a rate of 5.7 kg/day. The first extract stream at a rate of 4233 kg/day was recovered and combined with 1981 kg/day a secondary extract stream comprising recycle L-mannose from the second SMB zone to provide a combined first evaporization zone feed of 6214 kg/day and passed to a first evaporization zone. The combined feed to the first evaporization zone is shown in Table 3.

TABLE 3
Composition of First Evaporization Zone Feed

| | First | Second S. | Evap. |
|---|---|---|---|
| | Extract | Extract | Feed |
| Component | Wt-% | Wt-% | Wt-% |
| L-Mannose | 4.12 | 4.10 | 4.12 |
| L-Glucose | 2.21 | 0.11 | 1.51 |
| L-Arabinose | 0.15 | 0.27 | 0.10 |
| Na₂SO₄ | 0 | 0 | 5.60 |
| (NH₄)₂SO₄ | 0 | 0 | 0 |
| Pri Amines | 0 | 0 | 0 |
| Related | 0.5 | 0.05 | 0.01 |
| Monosaccharides | | | |
| Aldonic Acid | 0 | 0 | 0 |
| Water | 93.50 | 95.52 | 94.26 |
| Total | 100.00 | 100.00 | 100.00 |

The first evaporization zone removed 5026 kg/day of water and provided 1188 kg/day of evaporated isomerization zone feed. The isomerization effluent composition is shown in Table 4.

TABLE 4
Isomerization Effluent Composition

| Component | Isom. Effluent, Wt-% |
|---|---|
| L-Mannose | 9.72 |
| L-Glucose | 19.72 |
| L-Arabinose | 0.52 |
| Related | 0.5 |
| Monosaccharides | |
| Water | 70.00 |
| Total | 100.00 |

The isomerization effluent was passed to the second SMB zone. The second SMB zone contained 8 adsorbent beds in a 2-3-3 configuration, each adsorbent bed containing calcium exchange resin in the form of a spherical particle having a particle size of from 300 to 320 microns. The isomerization effluent and a second mobile phase desorbent stream comprising water was passed to the second SMB zone to provide 5392 kg/day of a second primary extract stream, 1989 kg/day of a second secondary extract stream, 24529 kg/day of a second desorbent effluent stream comprising water, and 7536 kg/day of a second raffinate stream. Table 5 shows the composition of the effluent streams from the second SMB zone.

TABLE 5
Second SMB Zone Effluent Streams

| | Second | Second | Second |
|---|---|---|---|
| | Primary | Sec. | Primary |
| | Extract | Extract | Raffinate |
| Component | Wt-% | Wt-% | Wt-% |
| L-Mannose | 0.63 | 4.10 | 0.03 |
| L-Glucose | 0.0 | 0.11 | 3.08 |
| L-Arabinose | 0.01 | 0.26 | 0.0 |
| Related | 0.5 | 0.0 | 0.0 |
| Monosaccharides | | | |
| Water | 99.35 | 95.53 | 96.89 |
| Total | 100.00 | 100.00 | 100.00 |

The second primary raffinate stream was passed to a second evaporization zone to at least a portion of the water to provide an overhead water stream of 7206 kg/day of water and 330.4 kg/day of a second evaporated raffinate stream having the composition shown in Table 6.

TABLE 6
Composition of Evaporated Second Raffinate

| | Second |
|---|---|
| | Evap. |
| | Raffinate |
| Component | Wt-% |
| L-Mannose | 0.71 |
| L-Glucose | 70.22 |
| L-Arabinose | 0.08 |
| Related Monosaccharides | 0.01 |
| Water | 28.98 |
| Total | 100.00 |

The second evaporated raffinate having a purity on a water free basis of about 99 wt-% L-glucose on a total sugar basis was passed to a crystallization and drying zone to form the high purity L-glucose product into a syrup, a granular or a crystalline product by conventional means.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims, while the description, abstract and drawings are not to be used to limit the scope of the invention. The invention is specifically intended to be as broad as the claims below and their equivalents.

What is claimed is:

1. A process for the production of a high purity L-glucose product from a mixed feed stream comprising L-glucose, L-mannose, salts and other sugars, said process comprising:
a. passing the mixed feed stream at a mixed feed temperature and a first mobile phase stream comprising water to an ion exclusion SMB zone comprising a plurality of ion exclusion beds, each of said ion exclusion beds containing an ion exclusion stationary phase agent comprising a strong acid sodium exchange resin and being selective for the adsorption of L-mannose and L-glucose and other sugars, said ion exclusion SMB zone being operated in an ion exclusion cycle to provide a first extract stream having a reduced concentration of salts and an initial concentration of L-glucose on a total sugar basis, said first extract stream comprising L-glucose, L-mannose, other sugars and water, a first primary raffinate stream comprising water and salts, and a first secondary raffinate stream comprising water;
b. admixing the first extract stream with a second secondary extract stream comprising L-mannose and water to provide an evaporization zone feed stream and passing the evaporization zone feed stream to an evaporization zone to provide an evaporization zone effluent stream comprising water, L-glucose and L-mannose and having a reduced concentration of water relative to the evaporization zone feed stream;
c. passing the evaporization zone effluent stream to an isomerization zone to at least partially transform a portion of the L-mannose into L-glucose to provide an isomerization zone effluent stream comprising L-glucose, L-mannose, and water, wherein the isomerization zone effluent stream has a concentration of L-glucose on a total sugar basis which is enhanced relative to said initial concentration of L-glucose in the first extract stream;
d. passing the isomerization zone effluent stream and a second mobile phase stream comprising water to a second SMB zone comprising a plurality of glucose separation beds, each of said glucose separation beds containing a glucose stationary phase agent comprising a strong acid calcium exchange resin being selective for the adsorption of L-glucose in a glucose adsorption cycle at effective glucose/mannose separation conditions to provide a second primary extract stream comprising L-mannose, a second secondary extract stream comprising L-arabinose, a second primary raffinate stream comprising high purity L-glucose, a second secondary raffinate stream comprising water; and
e) passing the second primary raffinate to a recovery zone to recover the high purity L-glucose product.

2. The process of claim 1, further comprising passing the second primary extract stream to be admixed with the first extract stream in step (b).

3. The process of claim 1, further comprising returning at least a portion of the first secondary raffinate stream comprising water to the ion exclusion SMB zone to provide at least a portion of the first mobile phase stream.

4. The process of claim 1, further comprising returning at least a portion of the second secondary raffinate stream comprising water to the second SMB zone to provide at least a portion of the second mobile phase stream.

5. The process of claim 1, wherein the recovery zone comprises:
a. passing the second primary raffinate stream to an evaporization zone comprising distillation or evaporization to provide an evaporated second raffinate stream; and
b. passing the evaporated second raffinate stream a crystallization and drying zone to provide the high purity L-glucose product.

6. The process of claim 1, wherein the high purity L-glucose product comprises from 90 to 99.9 wt-% of the an L-glucose based on the total sugar in said stream.

7. The process of claim 1, wherein the first mobile phase stream comprises deionized water.

8. The process of claim 1, wherein the at a mixed feed temperature ranges from about 40 to about 70° C.

9. The process of claim 1, wherein the ion exclusion cycle of the first SMB zone comprises a 2-3-2-1 SMB cycle having 2 ion exclusion beds in a desorption zone, 3 ion exclusion beds in a rectification zone, 2 ion exclusion beds in an adsorption zone, and 1 ion exclusion bed in a first regeneration zone.

10. The process of claim 1, wherein the ion exclusion SMB comprises 15 ion exclusion beds and the ion exclusion cycle of the first SMB zone comprises a 4-5-5-1 SMB cycle.

11. The process of claim 1, wherein the second SMB zone comprises a 1-1-3-2-1 SMB cycle having 1 adsorbent bed in a desorption zone, 1 adsorbent bed in a second desorption zone, 3 adsorbent beds in a rectification zone, and 2 adsorbent bed in an adsorption zone and 1 adsorbent bed in a first regeneration zone and 1 adsorbent bed in a second regeneration zone

12. The process of claim 1, wherein the second SMB zone comprises 15 adsorbent beds and the second SMB cycle comprises a 2-2-5-5-1 cycle.

13. A process for the production of a high purity L-glucose product from a mixed feed stream comprising L-glucose, L-mannose, salts and other sugars, said process comprising:
a. passing the mixed feed stream at a mixed feed temperature and a first mobile phase stream comprising water to an ion exclusion SMB zone comprising a plurality of ion exclusion beds, each of said ion exclusion beds containing an ion exclusion stationary phase agent comprising a strong acid sodium exchange resin and being selective for the adsorption of L-mannose and L-glucose and other sugars, said ion exclusion SMB zone being operated in an ion exclusion cycle to provide a first extract stream having a reduced concentration of salts and an initial concentration of L-glucose on a total sugar basis, said first extract stream comprising L-glucose, L-mannose, other sugars and water, a first primary raffinate stream comprising water and salts, and a first secondary raffinate stream comprising water;
b. admixing the first extract stream with a second secondary extract stream comprising L-mannose and water to provide an evaporization zone feed stream and passing the evaporization zone feed stream to an evaporization zone to provide an evaporization zone effluent stream comprising water, L-glucose and L-mannose and having a reduced concentration of water relative to the evaporization zone feed stream;
c. passing the evaporization zone effluent stream to an isomerization zone to at least partially transform a portion of the L-mannose into L-glucose to provide an isomerization zone effluent stream comprising L-glucose, L-mannose, and water, wherein the isomerization zone effluent stream has a concentration of L-glucose on a total sugar basis which is enhanced relative to said initial concentration of L-glucose in the first extract stream;
d. passing the isomerization zone effluent stream and a second mobile phase stream comprising water to a second SMB zone comprising a plurality of glucose separation beds, each of said glucose separation beds containing a glucose stationary phase agent comprising a strong acid calcium exchange resin being selective for the adsorption of L-glucose in a glucose adsorption cycle at effective glucose/mannose separation conditions to provide a second primary extract stream comprising L-mannose, a second secondary extract stream comprising L-arabinose, a second primary raffinate stream comprising high purity L-glucose, a second secondary raffinate stream comprising water;
e) passing the second primary raffinate to a recovery zone to recover the high purity L-glucose product;
f) passing the second primary extract stream to be admixed with the first extract stream in step (b);
g). returning at least a portion of the first secondary raffinate stream comprising water to the ion exclusion SMB zone to provide at least a portion of the first mobile phase stream;
h). returning at least a portion of the second secondary raffinate stream comprising water to the second SMB zone to provide at least a portion of the second mobile phase stream.
